(19) United States
(12) Patent Application Publication
(10) Pub. No.: US 2025/0375370 A1
(43) Pub. Date: Dec. 11, 2025

(54) PERSONAL CARE COMPOSITION, METHOD FOR USING SUCH COMPOSITIONS AND ITS IMPROVEMENT OF DISPERSIBILITY, STABILITY AND DEPOSITION EFFECTS

(71) Applicant: BASF SE, Ludwigshafen am Rhein (DE)

(72) Inventors: Ben Chuan ZHU, Shanghai (CN); Hsuan CHEN, Shanghai (TW); Hsin I. PENG, Shanghai (TW); Fang Qin LIN, Shanghai (CN)

(21) Appl. No.: 18/878,111

(22) PCT Filed: Jun. 21, 2023
(86) PCT No.: PCT/EP2023/066822

§ 371 Date: Dec. 23, 2024

(30) Foreign Application Priority Data
Jun. 28, 2022 (WO) PCT/CN2022/101923

(51) Int. Cl. A61K 8/60; A61K 8/37; A61Q 1/06; A61Q 5/02; A61Q 5/06; A61Q 5/12; A61Q 19/00; A61Q 19/10

(52) U.S. Cl. CPC A61K 8/60; A61K 8/375; A61Q 19/10; A61K 2800/805; A61Q 1/06; A61Q 5/02; A61Q 5/065; A61Q 5/12; A61Q 19/007

(57) ABSTRACT

The present invention relates to a cosmetic composition at least comprising a biosurfactant and a lipid layer enhancer. The present invention relates to a method of enhancing or facilitating a deposition of lipid layer enhancer compound onto keratinous materials, such as hair or skin.

### TECHNICAL FIELD

The present invention relates to a personal care composition, a method for using such composition and a method of facilitating or enhancing dispersibility, stability and deposition effect when the composition being applied onto keratinous materials.

### BACKGROUND ART

Personal care cleansing products can extract skin lipids besides removal of soil and dust from the skin. This may increase of skin roughness and transepidermal water loss. The negative effects will be even worsened by frequent treatment with surfactant-based rinse-off product.

In addition to considering usage of mild surfactant combinations to mitigate the skin roughness and transepidermal water loss, the blend of surfactant compatible lipid compounds is another efficient way to counteract negative effects on damage of skin lipid. Such blends will also enhance the deposition of lipid layer and offer conditioning/caring benefits.

Many personal care or cosmetic compositions comprise natural based, lipid layer enhancer which are applied to the skin and/or hair in a rinse-off formulation for conditioning effect. EP0776658 disclosed a mixture of the sebum-lipid glycerylmonooleate with mild alkyl polyglycosides. The mixture combines the moisturing properties of the lipid layer enhancer with the mildness of the biobased surfactant. The product can be simply formulated in the surfactant bases with cold process and also provides certain thickening benefits. Conditioning benefits, e.g., smoothness, are also widely claimed.

However, the viscosity of the blend is not stable but goes higher during storage. This will lead longer dispersing time during formulation. Reason behind might due to insufficient dispersibility of glyceryl monooleate in presence of alkyl polyglycoside. Another deficiency of such blend is insufficient deposition of lipid layer enhancer onto the keratinous materials, e.g. skin or hair.

However, it would be more challenging to obtain a surfactant-lipid layer enhancer combination which demonstrates improved dispersibility, including improved dispersibility and stability. The combination may also lead to improved of deposition of lipid layer enhancer onto the skin.

Sophorolipids are all-natural and consumer-friendly fermentation surfactant which are produced by several yeast species. In Candida bombicola, the hydrophilic moiety of the biosurfactant molecule is a disaccharide (i.e., sophorose), and the hydrophobic portion is an omega- or (omega-1)-hydroxy fatty acid attached to the sophorose via a glycosidic bond. The fatty acid chain, most commonly containing 16- and 18-carbon atoms, may be unsaturated and lactonized to the disaccharide. Sophorolipids, as biosurfactants, can serve as environmentally friendly surfactants being used widely in detergent and cosmetic formulations. EP 0550276 A1 describes a personal care composition comprising a mild and foaming glycolipid used with a co surfactant (e.g. SLS). WO 1998016192 A1 discloses a germicidal composition suitable for cleaning fruits, vegetables, skin and hair. The composition may comprise a mixture of anionic surfactant and sophorolipids biosurfactant. US patent application 2014/0349902 A1 discloses a hair and skin cleansing compositions comprising water, at least one biosurfactant and at least one fatty acid for obtaining an improved skin care benefit and foaming properties.

The present application has now discovered unexpectedly that a personal care composition containing a glycolipid biosurfactant and a lipid layer enhancer makes it possible to have improved dispersibility, stability and deposition onto the skin.

### SUMMARY OF INVENTION

The subject of the present invention is thus a personal care composition comprising at least:
a) a glycolipid biosurfactant, preferably sophorolipid biosurfactant;
b) a lipid layer enhancer, which is selected from the group consisting of esters of fatty acids of chain length C₈-C₂₄, preferably C₁₂-C₂₂ with glycerol.

Preferably, 10 to 90 wt. % of the sophorolipid biosurfactant is in the acidic form.

And preferably, the lipid layer enhancer is ester of glycerol of saturated or unsaturated C₈-C₂₄ fatty acids, preferably ester of glycerol of saturated or unsaturated C₁₂-C₂₂ fatty acids. And more preferably, the lipid layer enhancer is ester of glycerol of unsaturated C₁₂-C₂₂ fatty acids, Still more preferably, the lipid layer enhancer is ester of glycerol of unsaturated C₁₆-C₁₈ fatty acids. Still more preferably, the lipid layer enhancer is monoester of glycerol of unsaturated C₁₆-C₁₈ fatty acids

In a further aspect, the present invention is also directed toward a method of improving dispersibility and stability of a personal care composition comprising at least one biosurfactant and at least one lipid layer enhancer. In still a further aspect, the present invention is directed to a method for improving a deposition of lipid layer enhancer compound onto keratinous materials, comprising at least one step of applying to the keratinous materials an effective amount of the personal care composition of the present invention.

In another aspect, the present invention is also directed to use of the present personal care composition for improving dispersibility and stability, and enhancing deposition of lipid layer enhancer compound onto the skin.

### DETAILED DESCRIPTION OF THE INVENTION

Throughout the description, including the claims, the term “comprising one” or “comprising a” should be understood as being synonymous with the term “comprising at least one”, unless otherwise specified, and “between” should be understood as being inclusive of the limits.

The terms “a”, “an” and “the” are used to refer to one or to more than one (i.e., to at least one) of the grammatical object of the article.

The term “and/or” includes the meanings “and”, “or” and also all the other possible combinations of the elements connected to this term.

The term “rinse-off” used herein, refers to the composition are used in a context whereby the composition is ultimately rinsed or washed from the treated surface, (e.g. skin or hair) either after or during the application of the product.

The term “keratious material”, as used herein, means keratin-containing layers disposed as the outer layer of mammalian epidermal tissue, including skin, hair, nails, etc.

The term “lipid layer enhancer”, as used herein, means a functional ingredient which can prevent the skin from drying out.

It should be noted that in specifying any range of concentration, weight ratio or amount, any particular upper concentration, weight ratio or amount can be associated with any particular lower concentration, weight ratio or amount, respectively.

The present invention is directed to a personal care composition comprising at least:
a) a glycolipid biosurfactant, preferably sophorolipid biosurfactant;
b) a lipid layer enhancer compound, which is selected from the group consisting of esters of fatty acids of chain length C₈-C₂₄, preferably C₁₂-C₂₂ with glycerol.

The composition of the invention is a personal care composition or a cosmetic composition, preferably a personal care cleansing composition, more preferably a rinse-off cleansing composition, that is to say a composition used for the purpose of cleansing, conditioning, grooming, beautifying, or otherwise enhancing the appearance of the human body. Personal care products include skin care products, cosmetic products, antiperspirants, deodorants, perfume, toiletries, soaps, bath oils, feminine care products, hair-care products, oral hygiene products, depilatories, including shampoos, conditioners, hair straightening products and other haircare products, color cosmetics such as lipstick, creams, make-up, skin creams, lotions (preferably comprised of water-in-oil or oil-in-water emulsions), shave creams and gels, after-shave lotions and shave-conditioning compositions and sunscreen products, among numerous others. In preferred aspects, personal care compositions according to the present invention include haircare and skin care products, especially shampoos, conditioners, rinses, detangler products, hair color products, body washes, make-up, lipstick, skin creams and other skin-care products.

### Sophorolipid Biosurfactant

The composition of the present invention comprises at least one sophorolipid biosurfactant.

Biosurfactant is understood to be substances that are formed by microorganisms and are often expelled from the cell. Like classic surfactants, biosurfactant is surfact-active substance that reduce the surface tension of liquids and thereby promote the mixing of aqueous (hydrophilic) and water-repellent (hydrophobic) phases. Biosurfactant can be produced under gentle production conditions that require little energy. They are generally easily biodegradable and are very environmentally friendly. Moreover, they are not toxic, nor are any toxic byproducts produced during the production thereof. Carbohydrate, in particular sugar, e.g. glucose, and/or lipopholic carbon sources such as fats, oils, partical glycerides, fatty acids, fatty alcohols, long-chain saturated or unsaturated hydrocarbons, are used as raw materials for the microbial production of said biosurfactant. According to the present invention, the biosurfactant is preferably biosurfactant produced by fermentation.

Biosurfactants include glycolipids, lipopeptides, lipoproteins, fatty acids, phospholipids, neutral lipids and polymeric surfactants, which can all also be used in the present invention. Glycolipids that can be used in the present invention are compounds in which one or more monosaccharie units are glycosidically bonded to a lipid moiety. Examples of glycolipids as biosurfactants that can be used according to the invention are rhamnolipids, sophorolipids, mannosylerythritol lipids and trehalose lipids.

Sophorolipids are produced by fermentation using yeasts such as Candida Bombicola (also known as Torulopsis bombicola), Yarrowia lipolytica, Candida apicola (Torulopsis apicola) and Candida bogoriensis, by growing said yeasts on sugars, hydrocarbons, plant oils or mixture thereof. Sophorolipids have the following formulae (a) (lactone form) and (b) (free acid), the two forms typically being provided in a mixture,

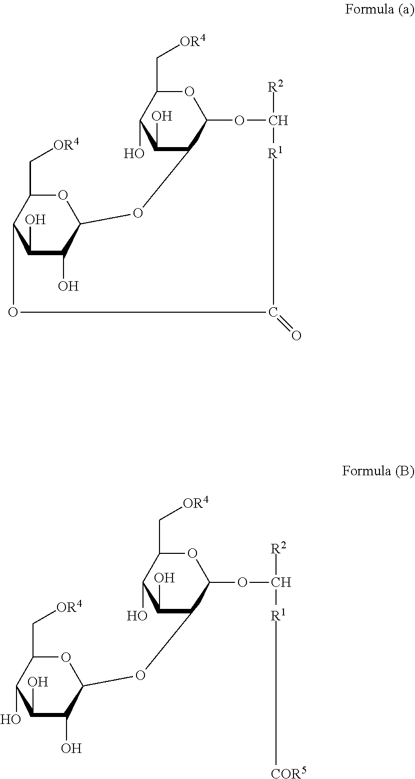

Where R¹ and R^{1′} independently represent saturated hydrocarbon chains or single or multiple, in particular single, unsaturated hydrocarbon chains having 8 to 20, in particular 12 to 18, carbon atoms, more preferably 14 to 18 carbon atoms, which can be linear or branched and can include one or more hydroxy groups, R² and R^{2′} independently represent a hydrogen atom or an acetyl group. Sophorolipid in which R¹ and R^{1′} are single, unsaturated, linear hydrocarbon chains having 15 carbon atoms are preferred. It is also preferred for R² and R^{2′} to represent a methyl group or a hydrogen atom or a saturated alkyl functional group or a single or multiple, in particular single, unsaturated alkyl functional group having 1 to 9 carbon atoms, which can be linear or branched and can include one or more hydroxy groups, and R³, R^{3′}, R⁴ and R^{4′} independently represent a hydrogen atom or an acetyl group, R⁵ represents hydroxy group or its form of salts.

According to the present invention, sophorolipids in which the acidic form and the lactone form in a mixture are preferred, preferably about 10 to about 90 wt. %, more preferably about 20 to about 60 wt. %, still more preferably about 25 wt. % to 40 wt. % of the sophorolipid being in acidic form and the remainder of the sophorolipid being in the lactone form.

Sophorolipids being suitable used for the present invention can be obtained commercially, for example and under the name BioToLife™ from BASF. The sophorolipid supplied by BASF, about 30 wt. % is present in the free acid form, in a mixture with the lactone form.

According to any one of the invention embodiments, the composition of the invention comprises from 0.1% to 30 wt. % of sophorolipid surfactant relative to the total weight of the composition, preferably from 0.2% to 20 wt. %, more particularly from 0.2% to 15 wt. %, still more preferably from 0.5% to 10 wt. %.

### Lipid Layer Enhancer Compound

The composition of the present invention comprises at least one lipid layer enhancer compound which is selected from the group consisting of esters of fatty acids of chain length C₈-C₂₄, preferably C₁₂-C₂₂ fatty acids with glycerol. Preferably, the lipid layer enhancer is ester of glycerol of saturated or unsaturated C₈-C₂₄ fatty acid, preferably of saturated or unsaturated C₁₂-C₂₂ fatty acids, and more preferably, the lipid layer enhancer is ester of glycerol of unsaturated C₁₂-C₂₂ fatty acids, still more preferably, the lipid layer enhancer is ester of glycerol of unsaturated C₁₆-C₁₈ fatty acids. Preference is given here to using monoesters of glycerol, monoesters of glycerol with unsaturated linear fatty acids in particular being suitable. Within the context of the invention, particular preference is given to glycerol monooleate. These glycerol esters are present in the present composition in amounts of from 4 to 10% by weight, preferably 5 to 9% by weight of the total weight of the composition.

According to any one embodiments of the present invention, the present invention is related to a personal care composition comprising at least one sophorolipid and at least one ester of glycerol of saturated or unsaturated C₈-C₂₄, preferably ester of glycerol of saturated or unsaturated C₁₂-C₂₂ fatty acids. In some preferred embodiment, the present invention is related to a personal care composition comprising at least one sophorolipid and at least one monoester of glycerol of unsaturated C₁₂-C₂₂ fatty acids. In some preferred embodiment, the present invention is related to a personal care composition comprising at least one sophorolipid and at least one ester of glycerol of unsaturated C₁₆-C₁₈ fatty acids. In some preferred embodiment, the present invention is related to a personal care composition comprising at least one sophorolipid and at least one glycerol monooleate.

According to any one embodiments of the present invention, the personal care composition comprises the glycolipid surfactant and the lipid layer enhancer in a weight ratio of 1:0.8 to 1:2, preferably of 1:1 to 1:2, more preferably of 1:1 to 1:1.5. In some preferred embodiments, the present invention is related to a personal care composition comprising at least one sophorolipid and at least one ester of glycerol of saturated or unsaturated C₈-C₂₄, preferably ester of glycerol of saturated or unsaturated C₁₂-C₂₂ fatty acids in a weight ratio of 1:0.8 to 1:2, preferably of 1:1 to 1:2, more preferably of 1:1 to 1:1.5. In some preferred embodiment, the present invention is related to a personal care composition comprising at least one sophorolipid and at least one monoester of glycerol of unsaturated C₁₂-C₂₂ fatty acids in a weight ratio of 1:0.8 to 1:2, preferably of 1:1 to 1:2, more preferably of 1:1 to 1:1.5. In some preferred embodiment, the present invention is related to a personal care composition comprising at least one sophorolipid and at least one monoester of glycerol of unsaturated C₁₆-C₁₈ fatty acids in a weight ratio of 1:0.8 to 1:2, preferably of 1:1 to 1:2, more preferably of 1:1 to 1:1.5. In some preferred embodiment, the present invention is related to a personal care composition comprising at least one sophorolipid and at least one glycerol monooleate in a weight ratio of 1:0.8 to 1:2, preferably of 1:1 to 1:2, more preferably of 1:1 to 1:1.5.

According to any one embodiments of the present invention, the sophorolipid used in the present personal care composition, is a mixture of acidic form and lactone form; preferably about 10 to about 90 wt. %, more preferably about 20 to about 60 wt. %, still more preferably about 25 wt. % to 40 wt. % of the sophorolipid being in acidic form and the remainder of the sophorolipid being in the lactone form.

### Additional Benefit Agent

The compositions of the present invention may further comprise one or more benefit agents that can provide a positive and/or beneficial effect to the substrate being cleaned, e.g. to the hair and skin. The skilled person is able to select according to general knowledge in the art of formulating personal care compositions such as shampoos, shower gels and liquid hand soaps, and the vast literature there-related, appropriate such optional ingredients for application purposes. In one embodiment, the composition of the present invention further comprises one or more benefit agents, such as emollients, moisturizers, conditioners, skin conditioners, or hair conditioners such as silicones such as volatile silicones, gums or oils, or non-amino silicones and mixtures thereof, mineral oils, esters, such as butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate, animal fats, including acetylated lanolin alcohols, lanolin, lard, mink oil and tallow, and fatty acids and alcohols, including behenic acid, palmitic acid, stearic acid, behenyl alcohol, cetyl alcohol, eicosanyl alcohol and isocetyl alcohol; vitamins or their derivatives, such as vitamin B complex, including thiamine, nicotinic acid, biotin, pantothenic acid, choline, riboflavin, vitamin B6, vitamin B12, pyridoxine, inositol, carnitine, vitamins A, C, D, E, K and their derivatives, such as vitamin A palmitate, and pro-vitamins, e.g., panthenol (pro vitamin B5), panthenol triacetate and mixtures thereof; antioxidants; free-radical scavengers; abrasives, natural or synthetic; dyes; hair coloring agents; bleaching agents; hair bleaching agents; UV absorbers, such as benzophenone, bornelone, PABA (Para Amino Benzoic Acid), butyl PABA, cinnamidopropyl trimethyl ammonium chloride, disodium distyrylbiphenyl disulfonate, potassium methoxycinnamate; anti-UV agents, such as butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, octyl dimethyl PABA (padimate O), red petrolatum; antimicrobial agents; antibacterial agents, such as bacitracin, erythromycin, triclosan, neomycin, tetracycline, chlortetracycline, benzethonium chloride, phenol, parachlorometa xylenol (PCMX), triclocarban (TCC), chlorhexidine gluconate (CHG), zinc pyrithione, selenium sulfide; antifungal agents; melanin regulators; tanning accelerators; depigmenting agents, such as retinoids such as retinol, kojic acid and its derivatives such as, for example, kojic dipalmitate, hydroquinone and its derivatives such as arbutin, transexamic acid, vitamins such as niacin, vitamin C and its derivatives, azelaic acid, placertia, licorice, extracts such as chamomile and green tea, where retinol, kojic acid, and hydroquinone are preferred; skin lightening agents such as hydroquinone, catechol and its derivatives, ascorbic acid and its derivatives; skin coloring agents, such as dihydroxyacetone; liporegulators; weight-reduction agents; anti-acne agents; anti-seborrhoeic agents; anti-ageing agents; anti-wrinkle agents; keratolytic agents; anti-inflammatory agents; anti-acne agents, such as tretinoin, isotretinoin, motretinide, adapalene, tazarotene, azelaic acid, retinol, salicylic acid, benzoyl peroxide, resorcinol, antibiotics such as tetracycline and isomers thereof, erythromycin, anti-inflammatory agents such as ibuprofen, naproxen, hetprofen, botanical extracts such as alnus, arnica, Artemisia capillaris, asiasarum root, calendula, chamomile, nidium, comfrey, fennel, galla rhois, hawthorn, houttuynia, hypericum, jujube, kiwi, licorice, magnolia, olive, peppermint, philodendron, salvia, sasa albomarginata, imidazoles such as ketoconazole and elubiol; refreshing agents; cicatrizing agents; vascular-protection agents; agents for the reduction of dandruff (Anti-dandruff agent), seborrheic dermatitis, or psoriasis, such as pyrithione salts, being formed from heavy metals such as zinc, tin, cadmium, magnesium aluminum, sodium and zirconium, like zinc pyrithione, shale oil and derivatives thereof such as sulfonated shale oil, selenium sulfide, sulfur, salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, ciclopirox olamine, anti-psoriasis agents such as vitamin D analogs, e.g. calcipotriol, calcitriol, and tacaleitrol, vitamin A analogs such as esters of vitamin A, including vitamin A palmitate, retinoids, retinols, and retinoic acid, corticosteroids such as hydrocortisone, clobetasone, butyrate, clobetasol propionate; antiperspirants or deodorants, such as aluminum chlorohydrates, aluminum zirconium chlorohydrates; immunomodulators; nourishing agents; depilating agents, such as calcium thioglycolate, magnesium thioglycolate, potassium thioglycolate, strontium thioglycolate; agents for combating hair loss; reducing agents for permanent-waving; reflectants, such as mica, alumina, calcium silicate, glycol dioleate, glycol distearate, silica, sodium magnesium fluorosilicate; essential oils and fragrances.

According to some invention embodiments, the composition of the present invention may further comprise polymeric or non-polymeric thickener to make the composition have satisfactory viscosity for use purpose. The composition may comprise less than 10 wt. % of an additional thickener. Suitable classes of thickeners for the present composition include, but are not limited to sodium chloride, potassium chloride, ammonium chloride, sodium sulfate, fatty acid alkylolamides, cellulose derivatives, carboxylic acid polymers, crosslinked polyacrylate polymers, polyacrylamide polymers, polysaccharides, gums and mixtures thereof. Non-limiting examples of suitable thickening agents are described in the CTFA International Cosmetic Ingredient Dictionary, 10th Ed. (2004), pp. 2294-96.

According to any one of the invention embodiments, the personal care composition is used in a manner know in the art, for example, in the case of a cleanser or shampoo, by application of the cleanser or shampoo to the skin and/or hair and optionally rinsing the cleanser or shampoo off of the skin and/or hair with water. According to any one of the invention embodiments, the composition of the invention may have a pH comprised between 4 and 11, for instance between 4 and 6.

The personal care composition of the present invention can be prepared by mixing individual components using any conventional blending technique known in the prior art such as conventional stirring, shaking or tumbling. These components may be supplied as concentrated solutions which are diluted and/or and combined in appropriate ratios by the skilled person. The invention covers any concentrate to be used as component ingredient to prepare a composition of the invention, and especially to concentrates containing limited levels of water due to some reasons from a cost and environmental perspective.

The present invention is also directed toward a method of improving dispersibility and stability of the personal care composition comprising at least one lipid layer enhancer as above described. The lipid layer enhancer can be used in combination with at least one sophorolipid to obtain an dispersion with improved dispersibility and stability.

The present invention is directed to a method of improving dispersibility and stability of a personal care composition comprising at least one lipid layer enhancer, wherein the method comprises combining the lipid layer enhancer with at least one glycolipid biosurfactant; preferably the lipid layer enhancer is esters of glycerol of saturated or unsaturated C₈-C₂₄ fatty acid, preferably of saturated or unsaturated C₁₂-C₂₂ fatty acids and the glycolipid biosurfactant is sophorolipid; more preferably the lipid layer enhancer is ester of glycerol of C₁₂-C₂₂ unsaturated linear fatty acids and the glycolipid biosurfactant is sophorolipid; still more preferably, the lipid layer enhancer is ester of glycerol of unsaturated C₁₆-C₁₈ fatty acids and the glycolipid biosurfactant is sophorolipid, further more preferably, the lipid layer enhancer is monoester of glycerol of C₁₆-C₁₈ unsaturated linear fatty acids and the glycolipid biosurfactant is sophorolipid; still further more preferably, the lipid layer enhancer is glycerol monooleate and the glycolipid biosurfactant is sophorolipid.

The present invention is directed to a method of improving dispersibility and stability of a personal care composition comprising at least one lipid layer enhancer, the method comprises combining the lipid layer enhancer with at least one glycolipid biosurfactant. The present invention is also directed toward a method of improving dispersibility and stability of a personal care composition comprising at least one ester of glycerol of unsaturated C₈-C₂₄ fatty acid, preferably of unsaturated C₁₂-C₂₂ fatty acids, wherein the method comprises combining the ester of glycerol of unsaturated C₁₂-C₂₂ fatty acids with at least one sophorolipid. The present invention is also directed toward a method of improving dispersibility and stability of a personal care composition comprising at least one monoester of glycerol of unsaturated C₁₂-C₂₂ fatty acids, preferably monoester of glycerol of unsaturated C₁₆-C₁₈ fatty acids, wherein the method comprises combining the monoester of glycerol of unsaturated C₁₂-C₂₂ fatty acids with at least one sophorolipid. The present invention is also directed toward a method of improving dispersibility and stability of a personal care composition comprising at least one glycerol monooleate, wherein the method comprises combining the glycerol monooleate with at least one sophorolipid.

The present invention is also directed toward a method of enhancing deposition of lipid layer enhancer onto the keratinous materials, which comprises at least one step of applying to the keratinous materials an effective amount of the personal care composition of the present invention comprising at least one sophorolipid and at least one ester of glycerol of unsaturated C₈-C₂₄ fatty acid, preferably of unsaturated C₁₂-C₂₂ fatty acids. The present invention is also directed toward a method of enhancing deposition of lipid layer enhancer onto the keratinous materials, which comprises at least one step of applying to the keratinous materials an effective amount of the personal care composition of the present invention comprising at least one sophorolipid and at least one monoester of glycerol of unsaturated C₁₂-C₂₂ fatty acids. The present invention is also directed toward a method of enhancing deposition of lipid layer enhancer onto the keratinous materials, which comprises at least one step of applying to the keratinous materials an effective amount of the personal care composition of the present invention comprising at least one sophorolipid and at least one glycerol monooleate.

According to any one embodiments of the present invention, the sophorolipid biosurfactant used in the above described method, is a mixture of acidic form and lactone form; preferably about 10 to about 90 wt. %, more preferably about 20 to about 60 wt. %, still more preferably about 25 wt. % to 40 wt. % of the sophorolipid being in acidic form and the remainder of the sophorolipid being in the lactone form.

### EXAMPLES

### Materials

| | | | Active |
|---|---|---|---|
| | | | matter |
| Ingredient | INCI | | (wt %) |
| Monomuls ® 90-O 18 | Glyceryl Oleate | Lipid layer | 100 |
| | | enhancer | |
| Monomuls ® 90-L 12 | Glyceryl Laurate | Lipid layer | 100 |
| | | enhancer | |
| Cutina ® GMS | Glyceryl Stearate | Lipid layer | 100 |
| | | enhancer | |
| Cegesoft ® HF 52 | Hydrogenated | Lipid layer | 100 |
| | Vegetable Oil | enhancer | |
| BioToLife ™ | Sophorolipid (Yeast | Surfactant | 30.0-50.0 |
| | Ferment Extract) | | |
| Plantapon ® Amino | Sodium Lauroyl | Surfactant | 100 |
| SLG-P | Glutamate | | |
| Plantacare ® 818 UP | Coco-Glucoside | Surfactant | 50.0-60.0 |
| Dehyton ® PK 45 | Cocamidopropyl | Surfactant | 44.0-46.0 |
| | Betaine | | |
| Texapon ® N 70 | Sodium Laureth | Surfactant | 60.0-80.0 |
| | Sulfate | | |

All the above listed materials are commercially available from BASF.

### I. Stability & Dispersibility Test:

Preparation of combination of biosurfactant and lipid layer enhancer
500 g of lipid layer enhancer (glycerol esters, active content) is heated above its melting point until fully melted.
500 g of pre-heated sophorolipid (active content) is then gradually added into melted lipid layer enhancer with continuous stirring at 100 rpm.
Deionized water is added to mixture to have the solid content at 65% based on the total weight of the composition under stirring for 30 minutes at 100 rpm until fully homogenized. A viscous dispersion is achieved after cooling down.

### Transmittance and Stability Measurement

The instrument used for transmittance and stability analysis is by LUMiSizer® 611 from LUM GmbH with radiation at wavelength of 865 nm and light factor of 1. The obtained combinations (prepared by the method described hereinabove) are diluted into 5 wt % and 0.4 ml of suspensions are filled into a rectangular polycarbonate synthetic cell with 2 mm optical path. The measurements were carried out at 25° C. and data was analyzed by the software SEPView® provided by LUM GmbH with below settings:
Rotational frequency—1000 rpm
Detection intervals—120×10 s
Measurement time—20 min
During the measurement, the software recorded the transmission in adjusted intervals. Initial transmission at 120 mm of the position over the cell length is recorded in below table.
The instability index was calculated by the software to quantitatively measure formulation stability in below table. It is a specific value in the range of 0-1, combing with Stokes' Law and Lambert-Beer's Law, demonstrating the relative value of the sume of transparency change to the maximum clarification during centrifugation.

The lower instability index indicates the higher stability performance. The higher transmission value indicates the more homogeneous appearance (higher dispersibility) observed.

### Test Results of Transmittance & Stability

| | | Transmission |
|---|---|---|
| | Instability | at 120 mm |
| Examples | Index | in % |
| Sample 1 (Combination of Monomuls ® | 0.09 | 49.74 |
| 90-O 18 and BioToLife ™, in weight | | |
| ratio of active content of1:1) | | |
| Sample 2 (Combination of Monomuls ® | 0.203 | 16.69 |
| 90-O 18 and Plantapon ® Amino SLG-P, | | |
| in weight ratio of active content of1:1) | | |
| Sample 3 (Combination of Monomuls ® | 0.392 | 58.33 |
| 90-O 18 and Texapon ® N 70, in | | |
| weight ratio of active content of 1:1) | | |
| Sample 4 (Combination of Monomuls ® | 0.002 | 16.69 |
| 90-O 18 and Dehyton ® PK 45, in | | |
| weight ratio of active content of 1:1) | | |
| Sample 5 (Combination of Monomuls ® | 0.071 | 33.72 |
| 90-O 18 and Plantacare ® 818 UP, in | | |
| weight ratio of active content of 1:1) | | |
| Sample 6 (Combination of Monomuls ® | 1 | 19.6 |
| 90-L 12 and Plantapon ® Amino SLG-P, | | |
| in weight ratio of active content of 1:1) | | |
| Sample 7 (Combination of Monomuls ® | 0.705 | 26.59 |
| 90-L 12 and BioToLife ™, in weight | | |
| ratio of active content of 1:1) | | |
| Sample 8 (Combination of Monomuls ® | 0.021 | 10.29 |
| 90-L 12 and Dehyton ® PK 45, in | | |
| weight ratio of active content of 1:1) | | |
| Sample 9 (Combination of Monomuls ® | 0.55 | 25.72 |
| 90-L 12 and Plantacare ® 818 UP, in | | |
| weight ratio of active content of 1:1) | | |
| Sample 10 (Combination of Monomuls ® | 1.12 | 73.61 |
| 90-L 12 and Texapon ® N 70, in weight | | |
| ratio of active content of 1:1) | | |
| Sample 11 (Combination of Cutina ® | 0.263 | 7.08 |
| GMS and Plantapon ® Amino SLG-P, | | |
| in weight ratio of active content of 1:1) | | |
| Sample 12 (Combination of Cutina ® | 0.007 | 6.06 |
| GMS and Plantacare ® 818 UP, in | | |
| weight ratio of active content of 1:1) | | |
| Sample 13 (Combination of Cutina ® | 0.385 | 5.92 |
| GMS and BioToLife ™, in weight | | |
| ratio of active content of 1:1) | | |
| Sample 14 (Combination of Cutina ® | 0.822 | 7.37 |
| GMS and Dehyton ® PK 45, in weight | | |
| ratio of active content of 1:1) | | |
| Sample 15 (Combination of Cutina ® | 0.707 | 6.94 |
| GMS and Texapon ® N 70, in weight | | |
| ratio of active content of 1:1) | | |
| Sample 16 (Combination of Cegesoft ® | 0.209 | 7.67 |
| HF 52 and Plantapon ® Amino SLG-P, | | |
| in weight ratio of active content of 1:1) | | |
| Sample 17 (Combination of Cegesoft ® | 0.002 | 4.9 |
| HF 52 and Plantacare ® 818 UP, in | | |
| weight ratio of active content of 1:1) | | |
| Sample 18 (Combination of Cegesoft ® | 0.138 | 8.39 |
| HF 52 and BioToLife ™, in weight ratio | | |
| of active content of 1:1) | | |
| Sample 19 (Combination of Cegesoft ® | 0 | 10.87 |
| HF 52 and Dehyton ® PK 45, in weight | | |
| ratio of active content of 1:1) | | |
| Sample 20 (Combination of Cegesoft ® | 0.023 | 6.65 |
| HF 52 and Texapon ® N 70, in weight | | |
| ratio of active content of 1:1) | | |

### II. Lipid Layer Enhancer Deposition Test:

### Formulation Details:

| | Formu- | Formu- | Formu- | Formu- | Formu- | Formu- |
|---|---|---|---|---|---|---|
| | lation 1 | lation 2 | lation 3 | lation 4 | lation 5 | lation 6 |
| | % by | % by | % by | % by | % by | % by |
| Ingredients | weight | weight | weight | weight | weight | weight |
| Texapon ® | 15 | 15 | 15 | 15 | 15 | 15 |
| N 70 | | | | | | |
| Dehyton ® | 6 | 6 | 6 | 6 | 6 | 6 |
| PK 45 | | | | | | |
| Sodium | 1 | 1 | 1 | 1 | 1 | 1 |
| Chloride | | | | | | |
| Sodium | 0.5 | 0.5 | 0.5 | 0.5 | 0.5 | 0.5 |
| Benzoate | | | | | | |
| Monomuls ® | / | 1.0 | 1.7 | 1.0 | 1.7 | 1.7 |
| 90-O 18 | | | | | | |
| BioToLife ™ | / | 2.5 | 4.3 | / | / | / |
| Plantacare ® | / | / | / | 1.8 | 3.1 | / |
| 818 UP | | | | | | |
| Water | To 100 | To 100 | To 100 | To 100 | To 100 | To 100 |

### Test Method Description:

### Pre-Rinsing:

Pre-rinsing the forearm with approximately 6 mL of a 10% Plantapon® 818UP solution (2 droppers full). Rubbing lightly in circular motion for 20 times.
Rinsing off with slow flowing DI water while rubbing in circular motion for 20 times. Dabbing dry lightly with paper towel.

### Applying Formulation:

Marking the corners of a 4 cm×10 cm area with a wax pen.
Applying 0.5 g of the rinse-off formulation onto the center of the area using a dose-controlled pipette.
Spreading immediately the 0.5 g rinse-off formulation in a circular motion for 20 times. Making sure the formulation was distributed evenly across the pre-marked 4 cm×10 cm area
Placing the forearm under slow flowing DI water right after the previous step and rubbing lightly under circular motion over 20 times. Removing the forearm from the water and dabbing dry lightly with paper towel.
Collecting Glycerol monooleate and measuring the deposition amount:
Using an alcohol swab to wipe approximately 20 times the test area to ensure thorough extraction of glycerol monooleate.
Transferring the alcohol swab to 5 mL of absolute alcohol and check concentration by liquid chromatography (using the parameters listed in below table). Results are presented as ug GMO in 10 cm² area.

| Instrument | Agilent 1260 HPLC coupled with 6224 TOF/MS | | |
|---|---|---|---|
| Column | Agilent Eclipse Plus C18, 3.0*150 mm, 3.5 μm | | |
| Temperature | 35° C. | | |
| Mobile phase | A: 5 mM ammonium acetate, | | |
| | 5% acetonitrile in water; | | |
| | B: Acetonitrile | | |
| | Time | Flow | |
| | (min) | (ml/min) | % B |
| Gradient | 0 | 0.6 | 75 |
| elution | 10 | 0.6 | 100 |
| | 12 | 0.6 | 100 |

| | |
|---|---|
| Equilibrium time | 5 min |
| Injection volume | 5 μl |
| Mass detector | ESI, negative; fragmentor: 110 V; scan mode: |
| | m/z 90-600, sim mode (EIC): m/z 415.3. |
| Retention time | Glycerol monooleate: 7.5 min, 8.1 min. |

### Test Results of the Deposition Amounts:

| | μg GMO/10 cm² |
|---|---|
| Samples | skin area |
| Formulation 1 | 0.62 |
| Formulation 2 | 8.25 |
| Formulation 3 | 16.39 |
| Formulation 4 | 4.00 |
| Formulation 5 | 9.84 |
| Formulation 6 | 4.67 |

The test results illustrate the deposition of glyceryl monooleate onto the skin is better and much improved when being used in combination with sophorolipid in a rinse-off personal care formulation.

1.-9. (canceled)

10. A personal care composition comprising at least:
a) a glycolipid biosurfactant and b) a lipid layer enhancer;
wherein the lipid layer enhancer is esters of glycerol of saturated or unsaturated C₈-C₂₄ fatty acids.

11. The composition of claim 10, wherein the glycolipid biosurfactant is sophorolipid.

12. The composition of claim 10, wherein the composition comprises at least one sophorolipid and at least one ester of glycerol of unsaturated C₁₂-C₂₂ fatty acids.

13. The composition of claim 11, wherein the sophorolipid biosurfactant is a mixture of acidic form and lactone form.

14. The composition of claim 13, wherein about 10 wt. % to about 90 wt. % of the sophorolipid is in acidic form and the remainder of the sophorolipid is in the lactone form.

15. The composition of claim 10, wherein the lipid layer enhancer is monoesters of glycerol of C₁₂-C₂₂ unsaturated linear fatty acids.

16. The composition of claim 15, wherein the monoesters of glycerol of C₁₂-C₂₂ unsaturated linear fatty acids is glycerol monooleate.

17. The composition of claim 10, wherein the glycolipid biosurfactant and the lipid layer enhancer are present in a weight ratio of 1:0.8 to 1:2.

18. The composition of claim 10, further comprising one or more anionic surfactants, nonionic surfactants or amphoteric surfactants.

19. The composition of claim 10, further comprising one or more additional benefit agents.

20. A method of improving dispersibility and stability of a personal care composition comprising at least one lipid layer enhancer, wherein the method comprises combining the lipid layer enhancer with at least one glycolipid biosurfactant.

21. The method of claim 20, wherein the lipid layer enhancer is esters of glycerol of saturated or unsaturated C₈-C₂₄ fatty acids.

22. The method of claim 20, wherein the lipid layer enhancer is ester of glycerol of C₁₂-C₂₂ unsaturated linear fatty acids and the glycolipid biosurfactant is sophorolipid.

23. The method of claim 20, wherein the lipid layer enhancer is monoester of glycerol of C₁₆-C₁₈ unsaturated linear fatty acids and the glycolipid biosurfactant is sophorolipid.

24. A method of enhancing deposition of lipid layer enhancer, which comprises at least one step of applying to the keratinous materials an effective amount of the personal care composition as defined according to claim 10, wherein the lipid layer enhancer is esters of glycerol of saturated or unsaturated C₈-C₂₄ fatty acids.

25. The method of claim 24, wherein the lipid layer enhancer is esters of glycerol of saturated or unsaturated C₁₂-C₂₂ fatty acids.

26. The method of claim 24, wherein composition comprises at least one sophorolipid and at least one ester of glycerol of unsaturated C₁₂-C₂₂ fatty acids.

27. The method of claim 24, wherein the lipid layer enhancer is monoesters of glycerol of C₁₆-C₁₈ unsaturated linear fatty acids.

28. The method of claim 24, wherein the lipid layer enhancer is glycerol monooleate.

29. A method of enhancing or facilitating a deposition of lipid layer enhancer onto keratinous materials, the method comprising using the personal care composition according to claim 10.
